(19) United States
(12) Patent Application Publication
(10) Pub. No.: US 2020/0337898 A1
(43) Pub. Date: Oct. 29, 2020

(54) EYE DROP MEDICATION KIT

(71) Applicant: ADVANCED INFUSION SOLUTIONS ACQUISITION, LLC, Ridgeland, MS (US)

(72) Inventor: Timothy E. Askew, Baltimore, MD (US)

(21) Appl. No.: 16/882,083

(22) Filed: May 22, 2020

Related U.S. Application Data
(60) Provisional application No. 62/822,483, filed on Mar. 22, 2019.

(51) Int. Cl. A61F 9/00

(52) U.S. Cl. CPC A61F 9/0026

(57) ABSTRACT

An eye drop medication kit with simplified SIG label and method of use for pre-operative and post-operative treatment using a simplified data-based representation of the desired dosage schedule incorporating a unique identifier system associated with a given dosage schedule and a desired eye drop formulation.

### CROSS-REFERENCE TO RELATED APPLICATIONS

This application claims priority to U.S. provisional application No. 62/822,483 titled “Eye Drop Medication Kit” and filed Mar. 22, 2019, the contents of which are incorporated herein by reference in its entirety.

### BACKGROUND

Prescription eye drops may be used to treat certain eye diseases and conditions, and are often prescribed as pre-operative and post-operative treatments for various eye surgeries. Complex pre-operative or post-operative eye drop treatment regimens can include applying several doses over time using multiple different eye drops. These treatment regimens are often accompanied by difficult to understand SIGs (i.e., signetur), otherwise known as “directions for use,” for the patient.

Complex directions for use decrease patient compliance with the prescribed treatments and ultimately lead to loss of efficacy for the medication being applied. Despite the widespread use of prescription eye drop medications, non-compliance due to complex directions for use is still an issue patients report to their prescribing physicians. Accordingly, a system and method that can effectively provide multiple eye drops to a patient in combination with easy to use instructions for a desired course of treatment that increases patient compliance would be useful.

### SUMMARY

The technology disclosed herein is directed to an eye drop medication kit and simplified SIG label or directions for use. In an example embodiment of the disclosed technology, an eye drop medication kit may include an eye drop bottle and SIG label comprising a treatment regimen, wherein the eye drop bottle comprises an eye drop formulation and a unique identifier that is specific to the eye drop formulation, and the treatment regimen comprises an indication of the unique identifier associating the treatment regimen with the eye drop bottle to be used. In some embodiments, the treatment regimen may comprise a start date for when the patient is to begin using the eye drop bottle. In some embodiments, the treatment regimen may include an end date for when the patient is to stop using the eye drop bottle. In some embodiments, the treatment regimen may include a dosage per day. The dosage per day, for example, may comprise an amount of times the eye drop bottle is to be used by the patient in a day.

In some embodiments, an eye drop medication kit may include one eye drop bottle. In some embodiments, an eye drop medication kit may include more than one eye drop bottle. In embodiments where an eye drop medication kit includes more than one eye drop bottle, each eye drop bottle may have its own eye drop formulation and its own unique identifier that is specific to the eye drop formulation. In embodiments, eye drop formulations may include serum eye drop formulations that are specific to a patient.

In embodiments, the unique identifier may comprise one or more of a color, a pattern, a symbol, a shape, and a texture. In some embodiments, the unique identifier may be a color. In some embodiments, the color comprises one or more of green, blue, red, black, yellow, orange, purple, brown, and white.

In embodiments, the SIG label may include machine readable labeling. Machine readable labeling may include, for example, a barcode or a quick response (“QR”) code. In some embodiments, the SIG label is adhesively attached to an inner top layer of the eye drop medication kit.

In embodiments, an eye drop medication kit may include an eye drop bottle, and a SIG label comprising a treatment regimen, wherein the eye drop bottle may include an eye drop formulation and a unique identifier that is specific to the eye drop formulation, and the treatment regimen may include an indication of the unique identifier associating the treatment regimen with the drop bottle to be used, a start date, an end date, and a dosage per day. In some embodiments, the treatment regimen may include more than one start dates. In some embodiments, the dosage per day may include a number indicating the amount of times the eye drop bottle is to be used by the patient in a day. In embodiments, the unique identifier may comprise one or more of a color, a pattern, a symbol, a shape, and a texture. In some embodiments, the unique identifier may be a color. In some embodiments, the color comprises one or more of green, blue, red, black, yellow, orange, purple, brown, and white. In embodiments, the SIG label may include machine readable labeling. Machine readable labeling may include, for example, a barcode or a quick response (“QR”) code.

In an example of the embodiments, a method for using an eye drop medication kit is disclosed. The eye drop medication kit, for example, may include an eye drop bottle comprising an eye drop formulation and a unique identifier that is specific to the eye drop formulation, and a SIG label comprising a treatment regimen, wherein the treatment regimen comprises an indication of the unique identifier, a start date, and end date, and a dosage per day. The method may include choosing an eye drop bottle based on the treatment regimen and applying the eye drop formulation to an eye in accordance with the treatment regimen. In some embodiments, choosing an eye drop bottle based on the treatment regimen may comprise identifying the indication.

In embodiments, the eye drop medication kit includes a SIG label. The SIG label may indicate, for example, one or more of the type of eye drop formulations to be used by the patient, the time or frequency or use of the eye drop formations, the amount of each given eye drop formulation to be used at a given time, the duration of a given course of treatment with the eye drop formulations and the order in which the eye drop formulations are to be applied. In some embodiments, the SIG label may be customized by a given physician for a patient. In some embodiments, the SIG label includes a display of a unique identifier that corresponds to an eye drop bottle and also may include to indicate a start date, and end date, a dose, and an order of using each eye drop formulation for both pre-operative treatment and post-operative treatment. In one embodiment, the SIG label may be provided in a format that is agnostic to a given language or even to the ability to read.

Other features and aspects of the disclosure will become apparent from the following detailed description, taken in conjunction with the accompanying drawings, which illustrate, by way of example, the features in accordance with various embodiments. The summary is not intended to limit the scope of the invention, which is defined solely by the claims attached hereto.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 illustrates, by way of example, a conventional representation of a SIG label.

FIG. 2 illustrates, by way of example, an eye drop medication kit in accordance with various embodiments of the disclosure.

FIG. 3 illustrates, by way of example, an eye drop medication kit in accordance with various embodiments of the disclosure.

FIG. 4 illustrates, by way of example, a clamshell enclosure in accordance with various embodiments of the disclosure.

FIG. 5 is an operational flow diagram illustrating an example method for using an eye drop medication kit in accordance with various embodiments of the disclosure.

The figures are not intended to be exhaustive or to limit the invention to the precise form disclosed. It should be understood that the invention can be practiced with modification and alteration, and that the disclosed technology be limited only by the claims and the equivalents thereof.

### DETAILED DESCRIPTION

Medicinal eye drops may be prescribed to treat various eye diseases and conditions, and are often prescribed before and after eye surgery to prevent infection and accelerate healing. Due to several advancements in the field of ophthalmology over the past decade, eye drop medications have grown increasingly complex. Indeed, multiple eye drop formulations may be prescribed to treat a certain eye condition, where often, each formulation is accompanied with difficult to follow SIG labels or directions for use. Difficult to follow instructions for use leads to lower patient compliance, resulting in loss of efficacy of the medication. To address the deficiencies of current eye drop medications and their directions for use, the technology disclosed herein is directed to an eye drop medication kit with a simplified SIG label that ensures patient compliance and allows for optimum efficacy of the eye drop medication prescribed.

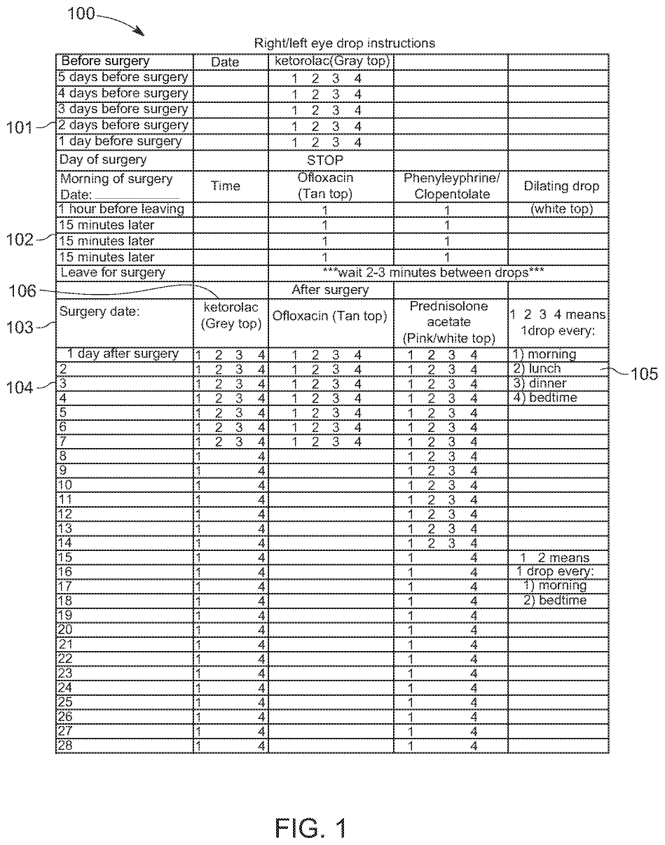

FIG. 1 illustrates, by way of example, a conventional SIG label or directions for use. The chart is complex and filled with difficult to follow instructions. For example, instruction chart 100 includes a pre-operative instruction section 101, a day of surgery instruction section 102 and a post-operative instruction section 103. Each section includes a multiple rows for each day or time of treatment, for example, the days after surgery 104. Each row consists of dosing times, in units of days, hours, and/or minutes, before surgery, after surgery, and the day of surgery, and has an associated dosing regimen indicated by a numbering system. These dosing regimens can be interpreted using an explanatory label or caption 105. A given dosing regimen is associated with a particular or specific medication 106. Instruction chart 100 is difficult to interpret and requires an adequate understanding of the language in which it is written. The difficulty associated with interpreting and using with instruction chart 100 of FIG. 1 adversely affects patient compliance and, therefore, decreases therapeutic results.

Various embodiments disclosed herein reformulate the conventional SIG label or directions for use into a simple system that is easily understood, does not require an ability to read a particular language or any language, accommodates patients with compromised visual acuity, minimizes the number of separate eye drop formulations to be used and facilitates or encourages standardization of pre-operative and post-operative treatments. This reformulation condenses or distills a conventional SIG label into as simple and concise a representation as possible while maintaining all desired medications and doses of medications within the original or convention SIG.

In various embodiments, the number of separate eye drop bottles may be minimized by combining multiple active ingredients into one bottle, or few bottles. Eye drop formulations containing multiple active ingredients may be delivered much more cost effectively, decreasing costs to the patient. In embodiments, customization of the SIG label for a given patient may be accomplished through changes in the numbers and types of medications or formulations provided, in the volumes of each medication included, and in colors or other indicia utilized to identify each medication or formulation. Customization of the SIG label may also include use of a barcode or quick response, i.e., a QR code that may be specific to a prescribing doctor or to a patient.

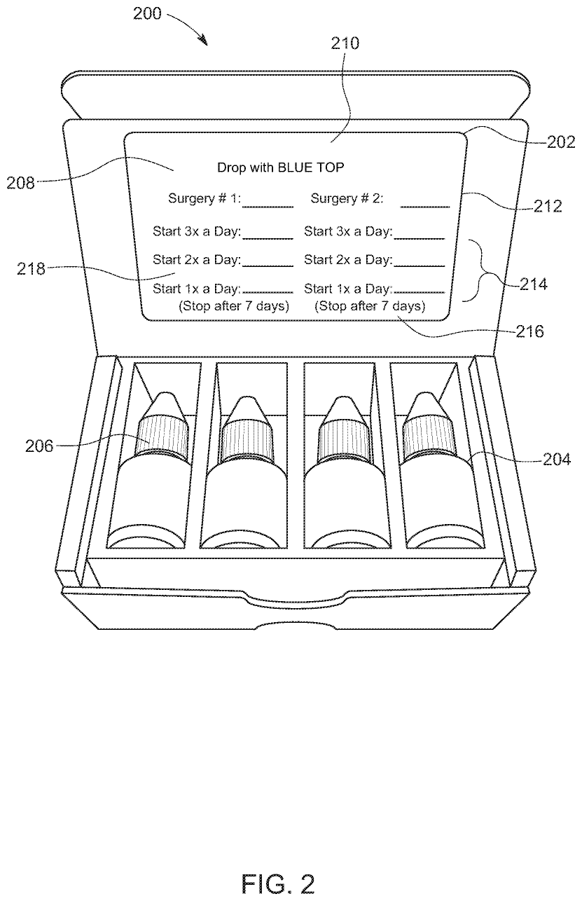

FIG. 2 illustrates by way of example, eye drop medication kit 200 in accordance with various embodiments of the disclosure. In embodiments, eye drop medication kit 200 may include SIG label 202, eye drop bottle 204 comprising unique identifier 206, treatment regimen 208 comprising indication 210, surgery date 212, a plurality of start dates 214, and end date 216.

In embodiments, eye drop medication kit 200 includes eye drop bottle 204. In some embodiments, eye drop medication kit 200 may include one eye drop bottle 204. In some embodiments, eye drop medication kit 200 may include more than one eye drop bottle 204. In embodiments, multiple eye drop formulations may be provided in more than one eye drop bottle 204. For example, eye drop medication kit 200 may include between 1-5 eye drop bottles 204. In embodiments, eye drop bottle 204 may include a distinct eye drop formulation. Distinct eye drop formulations may be used to treat different eye conditions, and/or may be used for pre-operation or post-operation procedures. In some embodiments, two or more of the eye drop bottles 204 can include the same or a therapeutically similar eye drop formulation. In embodiments, eye drop formulations may comprise serum eye drop formulations, antibiotic medications, pupil dilating formulations, eye lubricant formulations, as well as other active and inactive ingredient that may be included in such formulations.

In embodiments, eye drop bottle 204 may include a unique identifier 206. In some embodiments, eye drop bottle 204 may have more than one unique identifier 206. Unique identifier 206 may be located anywhere on eye drop bottle 204. In some embodiments, unique identifier 206 may be on the body of eye drop bottle 204. For example, in some embodiments, unique identifier 206 may be comprised of the eye drop bottle 204 itself, such as the color of the bottle. In some embodiments, unique identifier may be on a label attached to the eye drop bottle 204. In some embodiments, unique identifier 206 is on the cap of the eye drop bottle 204. For example, the unique identifier 206 may be the color of the cap.

In some embodiments, unique identifier 206 may include, for example, a color, a pattern, a symbol, an alpha-numeric character, a shape, a texture, and a tactile identifier. In some embodiments, more than one unique identifier 206 may be used. For example, more than one color may be unique identifier 206. In some embodiments, unique identifier 206 may include more than one pattern. In embodiments, eye drop medication kit 200 may include more than one eye drop bottle 204 having the same unique identifier 206. In some embodiments, eye drop bottle 204 may include printed information located on the body of eye drop bottle 204.

In various embodiments, eye drop medication kit 200 also includes SIG label 202. In embodiments, SIG label 202 may be attached, for example, to the interior of eye drop medication kit 200. In embodiments, SIG label 202 may be attached to the exterior of eye drop medication kit 200. In embodiments, SIG label 202 may include a machine readable object, for example, a barcode. In some embodiments, the machine readable object may be a quick response code (i.e., “QR code”). The machine readable object can be scanned and/or interpreted using, for example, a computer or smartphone. In embodiments, interpretation of the machine readable code can be used to link the eye drop medication kit to an application on a smart phone or to an existing calendar. In some embodiments, the application or calendar is auto-populated with a certain treatment regimen and can use the smartphone or other computer based functions to provide audio, visual or tactile reminders to the patient regarding the treatment scheme as identified on the SIG label. In some embodiments, SIG label 202 may be downloaded and displayed on the smart phone or other computing device. In embodiments, the application or smartphone may be linked to the doctor or other health care professionals, for example, assisted living facility staff or family members, to provide monitoring or feed-back on compliance with the prescribed treatment regimen or to provide for refills of prescribed eye drop formulations. In some embodiments, a computer readable code, smart phone, or other computing device may be used to link the SIG label 202 and an associated treatment regime to artificial intelligence systems and virtual assistants that are used to assist the patient in remembering to follow the prescribed treatment regimen. In embodiments, the QR code may be customized for a specific patient and/or healthcare provider, such that the SIG label (including the treatment regimen therein) in tailored to the specific patient and/or healthcare provider.

In embodiments, SIG label 202 includes treatment regimen 208. In some embodiments, SIG label 202 may include one treatment regimen 208. In some embodiments SIG label 202 may include more than one treatment regimen 208. In embodiments, treatment regimen 208 may be associated with a specific eye drop bottle 204. In some embodiments, more than one treatment regimen 208 is provided for a given eye drop bottle 204. In some embodiments, a treatment regimen is associated with two or more eye drop bottles 204.

In embodiments, treatment regimen 208 may include indication 210. In embodiments, indication 210 associates treatment regimen 208 with eye drop bottle 204 through unique identifier 206. For example, indication 210 may display unique identifier 206 such that unique identifier 206 may be used to associate the eye drop bottle 204 comprising the same unique identifier 206 to a particular treatment regimen 208. In some embodiments, indication 210 may be used to associate more than one eye drop bottle 204 with a treatment regimen 208. In embodiments, indication 210 may include, for example, the same aspects of unique identifier 206, including for example, a color, a texture and/or a shape. In embodiments, indication 210 may be visual, such as for example, a color, texture, pattern, number, and/or shape. In some embodiments, indication 210 may be tactile, such as a shape or raised texture.

In embodiments, indication 210 may be incorporated into treatment regimen 208. For example, treatment regimen 208 may include indication 210, wherein indication 210 is a color corresponding to the color of the unique identifier 206 on eye dropper bottle 204. In embodiments, all of treatment regimen 208 may comprise indication 210 such that all words of treatment regimen 208 are one color and may be used by the patient to associate treatment regimen 208 with the same color of unique identifier 206 on eye drop bottle 204. In some embodiments, indication 210 may only comprise a portion of treatment regimen 208, wherein only a portion of the treatment regimen 208 is in the corresponding color of a particular unique identifier 206. In some embodiments, treatment regimen 208 may include indication 210, wherein indication 210 is a specific shape. The same shape may be used by unique identifier 206 such that the patient can identify which eye drop bottle 204 corresponds to the treatment regimen 208 comprising an indication 210 with that shape.

In embodiments, treatment regimen 208 may also include surgery date 212. In embodiments, surgery date 212 may comprise the calendar date the surgery is to be performed. In embodiments, treatment regimen 208 may also comprise start date 214. In embodiments, start date 214 may comprise the calendar date for when the patient is to begin using eye drop bottle 204. The date on which the patent beings using the corresponding bottle is to be continued until the next start date or when indicated by an end date. In embodiments, treatment regimen 208 may comprise one start date 214. In some embodiments, treatment regimen 208 may comprise more than one start date 214. In some embodiments, start date 214 may be a date before surgery date 212. In some embodiments, start date 214 may be a date after surgery date 212. In embodiments, treatment regimen 208 may also include an end date 216. In some embodiments, end date 216 is the calendar date for when the patient is to stop using eye drop bottle 204. In some embodiments, end date 216 may be a date before surgery date 212. In some embodiments, end date 216 may be a date after surgery date 212.

In various embodiments, treatment regimen 208 may also comprise dosage per day 218. In embodiments, dosage per day 218 may comprise the amount (e.g., number) of times the eye drop bottle is to be used by the patient in a day. In some embodiments, dosage per day 218 may include a symbol, including, for example, the letter “X,” which herein means “times.” For example, dosage per day 218 may comprise “3×,” indicating that eye drop bottle 204 is to be used three (3) times in a day. In some embodiments, dosage per day 218 may comprise “2×,” indicating that eye drop bottle 204 is to be used two (2) times in a day. It is to be understood that any amount (i.e., number proceeding the “x”) may be used. A person of ordinary skill in the art would understand that the amount indicated by dosage per day 218 comprises an effective treatment amount, which may vary based on the formulation of the eye drop bottle 204 and the patient receiving the eye drop formulation.

In embodiments, surgery date 212, start date 214, and/or end date 216 may be depicted in the MM-DD-YYYY format, DD-MM-YYYY format or YYYY-MM-DD format.

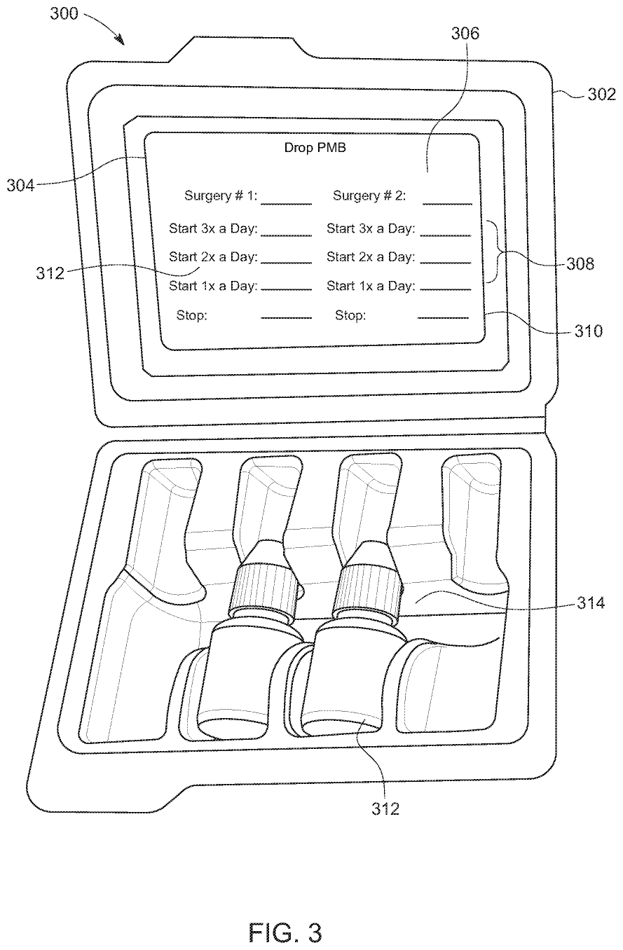

FIG. 3 illustrates by way of example, eye drop medication kit 300 in accordance with various embodiments of the disclosure. In embodiments, eye drop medication kit 300 may include a plastic clamshell 302 enclosure. In embodiments, eye drop medication kit 300 may include SIG label 304, wherein SIG label 304 may be mounted on the interior topside of the plastic clamshell 302. In some embodiments, SIG label 304 may include the components and content described herein, including one or more treatment regimen 306 comprising multiple start dates 308, an end date 310, and a dosage per day 312. As illustrated in FIG. 3, eye drop medication kit 300 may also include more than one eye drop bottle 312 containing the desired eye drop formulations with desired active ingredients. In embodiments, eye drop bottle 312 includes a unique identifier 314 to associate each bottle with a given treatment regimen 306.

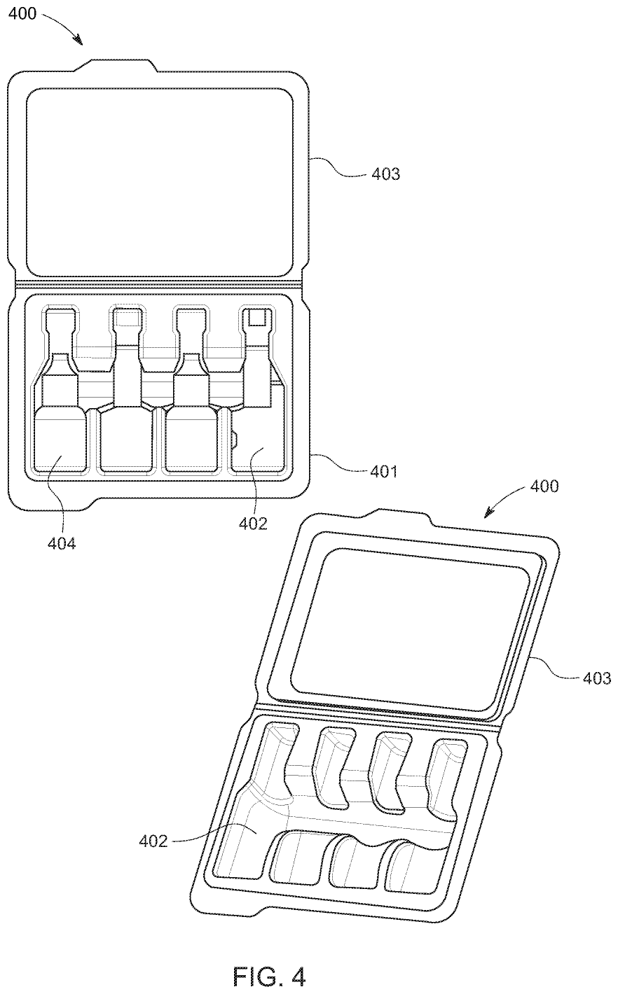

FIG. 4 illustrates by way of example, clamshell enclosure 400 that may be used by the various systems and methods of the disclosure. In embodiments, clamshell enclosure 400 provides a convenient way to transport, carry, and/or store the eye drops and associated SIG label. In embodiments, clamshell enclosure 400 may comprise dimensions of about 3 inches by about 5 inches. In embodiments, clamshell enclosure 400 may comprise dimensions of about 2 inches by about 4 inches. In embodiments, clamshell enclosure 400 may comprise dimensions of about 4 inches by 6 inches.

In embodiments, clamshell enclosure 400 may include a top 403 and a molded bottom 401 that is hinged to top 403. In some embodiments, molded bottom 401 may be shaped to hold eye drop bottle 404 in form fitting cavity 402. In some embodiments, more than one eye drop bottles 404 may be held by more than one form fitting cavity 402. In some embodiments, form fitting cavity 402 is configured so that each eye drop bottle 404 may “snap” into place and be held securely within clamshell enclosure 400. Clamshell enclosure 400 may be comprised of plastic. Several types of plastic may be used to manufacture clamshell enclosure 400 including but not limited to, acrylic, polyethylene terephthalate (PET), polystyrene and/or polycarbonate.

While illustrated with eye drop bottles and eye drops, systems and methods in accordance with the present invention can also be used to dispense other types of medicines. In one embodiment, medication kits can be provided that assist a person in taking all of the prescribed medications that the patient is taking. Exemplary embodiments are also directed to methods for producing the eye drop medication kit and methods for using the eye drop medication kit.

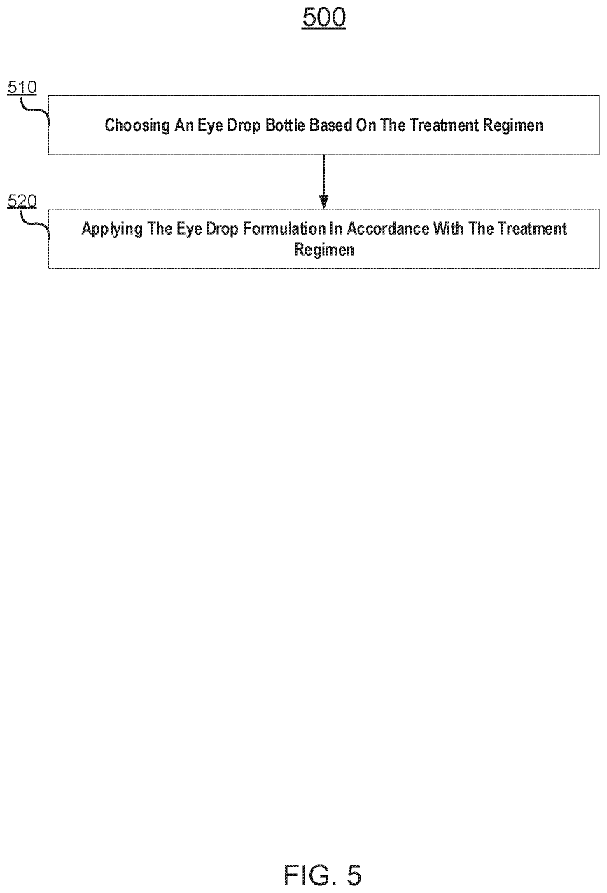

FIG. 5 is a flow diagram illustrating an example method in accordance with the technology disclosed. At a high level, method 500 may be performed to use an eye drop medication kit in accordance with various embodiments of the disclosure. The operations of the various methods described herein are not necessarily limited to the order described or shown in the figures, and one of skill in the art will appreciate, upon studying the present disclosure, variations of the order of the operations described herein that are within the spirit and scope of the disclosure. Let it be appreciated that operations of method 500 may be performed multiple times.

The operations and sub-operations of method 500 may be carried out, in some cases, using one or more of the components, elements, devices, and sub-components of eye drop medication kit 200, and as described with respect to at least FIGS. 2-4, as well as components, elements, devices, and sub-components, depicted therein and/or described with respect thereto.

In such instances, the description of method 500 may or may not refer to a corresponding component and/or element, but regardless of whether an explicit reference is made, one of skill in the art will recognize, upon studying the present disclosure, when the corresponding component and/or element may be used. Further, it will be appreciated that such references do not necessarily limit the described method to the particular component and/or element referred to. Thus, it will be appreciated by one of skill in the art that aspects and features described above in connection with (sub-) components, elements, devices, and components, including variations thereof, may be applied to the various operations described in connection with method 1000 without departing from the scope of the present disclosure.

Referring now to FIG. 5, method 500 may be implemented to use an eye drop medication kit in accordance with implementations of the disclosure. At operation 510, a patient may choose an eye drop bottle based on the treatment regimen. In embodiments, choosing an eye drop bottle based on the treatment regimen may comprise identifying an indication of the treatment regimen. For example, treatment regimen 208 may include indication 210, wherein indication 210 is a color. In embodiments, the color of indication 210 may be blue, while at the same time, unique identifier 206 of eye drop bottle 204 may also be blue. Based on the blue color of indication 210, a user will associate the treatment regimen 208 with the blue unique identifier 206 and choose eye drop bottle 204 that has unique identifier 206 that is blue. In some embodiments, indication 210 may be a shape or a raised texture, such that a user can associate treatment regimen 208 with that indication 210 and choose the corresponding eye drop bottle 204 based on its unique identifier 206 with the same shape or raised texture.

At operation 520, a patient may apply the eye drop formulation within the eye drop bottle chosen in accordance with the treatment regimen. In embodiments, once a patient chooses the correct eye drop bottle based on identifying the indication of the treatment regimen, the patient may apply the eye drop formulation to one or more eyes in accordance with the various components of the treatment regimen, including, the start date, the end date, and the dosage per day. In embodiments, a patient may apply the eye drop formulation within eye drop bottle 204 based on treatment regimen 208. In some embodiments, treatment regimen 208 may include surgery date 212, start date 214, end date 216, and dosage per day 218.

Although described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the application, whether or not such embodiments are described and whether or not such features are presented as being a part of a described embodiment. Thus, the breadth and scope of the present application should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The use of the term “component” does not imply that the components or functionality described or claimed as part of the component are all configured in a common package. Indeed, any or all of the various components of a component, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

The details of some embodiments of the systems and methods of the present disclosure are set forth in this description and in some cases, in other portions of the disclosure. Other features, objects, and advantages of the disclosure will be apparent to one of skill in the art upon examination of the present disclosure, description, figures, examples, and claims. It is intended that all such additional systems, methods, devices, features, and advantages be included within this description (whether explicitly or by reference), be within the scope of the present disclosure, and be protected by one or more of the accompanying claims.

What is claimed is:

1. An eye drop medication kit comprising:
an eye drop bottle; and
a SIG label comprising a treatment regimen;
wherein the eye drop bottle comprises an eye drop formulation and a unique identifier that is specific to an eye drop formulation; and
the treatment regimen comprises an indication of the unique identifier associating the treatment regimen with the eye drop bottle to be used.

2. The eye drop medication kit of claim 1, wherein the treatment regimen further comprises a start date for when the patient is to begin using the eye drop bottle.

3. The eye drop medication kit of claim 1, wherein the treatment regimen further comprises an end date for when the patient is to stop using the eye drop bottle.

4. The eye drop medication kit of claim 1, wherein the treatment regimen further comprises a dosage per day.

5. The eye drop medication kit of claim 4, wherein the dosage per day comprises an amount of times the eye drop bottle is to be used by the patient in a day.

6. The eye drop medication kit of claim 1, wherein the unique identifier comprises one or more of a color, a pattern, a symbol, a shape, and a texture. The eye drop medication kit of claim 6, wherein the unique identifier is a color.

8. The eye drop medication kit of claim 7, wherein the color comprises one or more of green, blue, red, black, yellow, orange, purple, brown, and white.

9. The eye drop medication kit of claim 1, wherein the SIG label further comprises machine readable labeling.

10. The eye drop medication kit of claim 9, wherein machine readable labeling comprises one or more of a barcode and a QR code.

11. The eye drop medication kit of claim 1, wherein the SIG label is adhesively attached to an inner top layer of the eye drop medication kit.

12. An eye drop medication kit comprising:
an eye drop bottle; and
a SIG label comprising a treatment regimen;
wherein the eye drop bottle comprises an eye drop formulation and a unique identifier that is specific to the eye drop formulation; and
the treatment regimen comprises an indication of the unique identifier associating the treatment regimen with the eye drop bottle to be used, a start date, an end date, and a dosage per day.

13. The eye drop medication kit of claim 12, wherein the dosage per day comprises an amount of times the eye drop bottle is to be used by the patient in a day.

14. The eye drop medication kit of claim 12, wherein the unique identifier comprises one or more of a color, a pattern, a symbol, a shape, and a texture.

15. The eye drop medication kit of claim 15, wherein the unique identifier comprises a color.

16. The eye drop medication kit of claim 16, wherein the color comprises one or more of green, blue, red, black, yellow, orange, purple, brown, and white.

17. The eye drop medication kit of claim 12, wherein the SIG label further comprises machine readable labeling.

18. The eye drop medication kit of claim 17, wherein machine readable labeling comprises one or more of a barcode and a QR code.

19. A method of using an eye drop medication kit comprised of an eye drop bottle comprising an eye drop formulation and a unique identifier that is specific to the eye drop formulation, and a SIG label comprising a treatment regimen, wherein the treatment regimen comprises an indication of the unique identifier, a start date, an end date, and a dosage per day, the method comprising:
choosing an eye drop bottle based on the treatment regimen; and
applying the eye drop formulation to an eye in accordance with the treatment regimen.

20. The method of claim 19, wherein choosing an eye drop bottle based on the treatment regimen comprises identifying the indication.
